(19) United States
(12) Patent Application Publication
(10) Pub. No.: US 2008/0233131 A1
(43) Pub. Date: Sep. 25, 2008

(54) Vaccine

(76) Inventor: Richard John Stebbings, Potters Bar (GB)

Correspondence Address: FULBRIGHT & JAWORSKI, LLP, 1301 MCKINNEY, SUITE 5100, HOUSTON, TX, 77010-3095, US

(21) Appl. No.: 11/575,280

(22) PCT Filed: Sep. 13, 2005
(86) PCT No.: PCT/GB2005/003530

§ 371 Date: Feb. 18, 2008

Related U.S. Application Data
(60) Provisional application No. 60/609,256, filed on Sep. 14, 2004.

(51) Int. Cl. A61K 39/395; A61K 39/00; A61P 37/00

(52) U.S. Cl. 424/172.1; 424/184.1

(57) ABSTRACT

The invention provides a method for generating anti-CD4 auto-antibodies comprising administering to an individual an anti-CD4 antibody molecule which has a low affinity for native CD4 in said individual or a protein or peptide comprising a CD4-like epitope, wherein administration of said antibody molecule, protein or peptide results in the generation of anti-CD4 auto-antibodies.

### FIELD OF THE INVENTION

The present invention relates to a method for generating anti-CD4 auto-antibodies in an individual. Methods for treating and/or preventing HIV infection and a pharmaceutical or vaccine composition are also provided.

### BACKGROUND TO THE INVENTION

Twenty years after HIV was identified, there is still no consensus over the immune responses a vaccine must elicit to prevent HIV infection. Clearly a vaccine is needed to halt the inexorable spread of the AIDS pandemic. Most current AIDS vaccine strategies are based on eliciting humoural or cellular immune responses. Such approaches have not proved effective at preventing infection and, with each negative vaccine report, there is growing concern that current strategies for AIDS vaccines under clinical development will not prove effective.

From simian models of HIV/AIDS, it is known that vaccination with live attenuated HIV elicits the most potent durable protection against infection and disease of any approach tested to date. This protection is not only effective against a diverse range of variants and multiple routes of infection but, despite an absence of HIV-1 neutralising antibodies, is also able to protect against chimeric SIV/HIV (SHIV) viruses in which the envelope (tat, vpu and rev genes) of SIV have been replaced with those of HIV. However, safety concerns as to the stability of mutations in attenuated viruses have precluded the clinical evaluation of live attenuated HIV vaccines.

Investigations seeking to understand the mechanism of protection conferred by live attenuated vaccines indicate that non-immune receptor blockade is central to the protective mechanism. A number of new anti-retroviral drugs in pre-clinical development work by inhibiting binding of HIV to primary and secondary receptors.

### SUMMARY OF THE INVENTION

The present inventors have identified a novel approach for receptor blockade based on the generation of anti-CD4 auto-antibodies. More specifically, the inventors have elicited anti-CD4 auto-antibodies in macaques by administering human anti-CD4 antibodies that mask the HIV/SIV receptor. The human antibodies have been mutated in the Fc region to prevent complement fixation and antibody dependent cellular cytoxicity (ADCC) that would otherwise cause cell lysis. The macaques have been challenged with SIV to test this new mechanism of protection.

Thus, the present invention provides a novel approach for preventing HIV infection by vaccination to elicit CD4-masking auto-antibodies. This novel approach can also be used in the therapeutic treatment of already infected vaccinates. Vaccines provided by the present invention have global efficacy since different envelope clades of HIV all over the world all use the same primary receptor, CD4.

Accordingly, the present invention provides:
a method of inhibiting entry of HIV into human cells, said method comprising administering to a human individual an anti-CD4 antibody molecule in an amount effective to elicit an anti-CD4 auto-antibody response in said individual, wherein binding of said anti-CD4 auto-antibodies to CD4 blocks the binding site for HIV;
a method of inhibiting entry of HIV into human cells, said method comprising administering to a human individual a protein or peptide comprising a human CD4-like epitope in an amount effective to elicit an anti-CD4 auto-antibody response in said individual, wherein binding of said anti-CD4 auto-antibodies to CD4 blocks the binding site for HIV.
a method of generating anti-CD4 auto-antibodies in a human individual comprising administering to an individual an anti-CD4 antibody molecule directed to a human CD4-like epitope, or a protein or peptide comprising a human CD4-like epitope, wherein administration of said antibody molecule, protein or peptide results in the generation of anti-CD4 auto-antibodies that block binding of HIV to CD4.
an anti-CD4 auto-antibody obtained or obtainable by a method according to the invention;
use of an anti-CD4 autoantibody according to the invention to inhibit binding of HIV to CD4;
a pharmaceutical or vaccine composition for administration to a human individual, which composition comprises an anti-CD4 antibody molecule directed to a human CD4-like epitope, or a protein or peptide comprising a CD4-like epitope, and a pharmaceutically acceptable carrier or diluent;
use of an anti-CD4 antibody molecule as defined herein in the manufacture of a medicament for the treatment or prophylaxis of HIV infection in an individual;
a method of vaccinating against HIV comprising administering to a human individual an anti-CD4 antibody molecule, or a protein or peptide comprising a CD4-like epitope, in an amount effective to elicit an anti-CD4 auto-antibody response in said individual, wherein binding of said anti-CD4 auto-antibodies to CD4 blocks the binding site for HIV;
a method of preventing the onset of AIDS in a human individual infected with HIV, comprising administering to a human individual an anti-CD4 antibody molecule or a protein or peptide comprising a human CD4-like epitope in an amount effective to elicit an anti-CD4 auto-antibody response in said individual, wherein binding of said anti-CD4 auto-antibodies to CD4 blocks the binding site for HIV;
a method of inhibiting entry of SIV or SHIV into primate cells, comprising administering to a primate individual an anti-CD4 antibody molecule in an amount effective to elicit an anti-CD4 auto-antibody response in said individual, wherein binding of said anti-CD4 auto-antibodies to CD4 blocks the binding site for SIV or SHIV; and
a method of inhibiting entry of SIV or SHIV into primate cells, said method comprising administering to a primate individual a protein or peptide comprising a primate CD4-like epitope in an amount effective to elicit an anti-CD4 auto-antibody response in said individual, wherein binding of said anti-CD4 auto-antibodies to CD4 blocks the binding site for SIV or SHIV.

### BRIEF DESCRIPTION OF THE FIGURES

FIG. 1 shows the inhibition of CD4 staining with the anti-CD4 monoclonal antibody SK3 observed in macaques challenged with pathogenic SIVmacJ5M following treatment with the humanized anti-human CD4 monoclonal antibody TRX1.

FIG. 2 shows that macaque CD4 auto-antibodies generated in response to TRX1 do not cause lysis of CD4 lymphocytes.

FIG. 3 shows the results of FACS analysis demonstrating that pre-incubation of naïve PBMC with serum from TRX1 treated macaques inhibited subsequent binding of CD4 monoclonal antibodies. PBMC from naïve macaques B1 to B4 were pre-incubated with serum collected from HIVIG (Human intravenous immunoglobulin) treated macaques A367 to A370 and anti-CD4 mAb treated macaques A363 to A366 for 5 minutes prior to counterstaining with the anti-CD4 mAb OKT4-FITC conjugate and SK3-PE conjugate. Pre-incubation serum was collected from macaques 56 days after HIVIG or anti-CD4 treatment. The anti-CD4 mAb OKT4 recognizes an epitope of CD4 not involved in HIV/SIV binding, whereas SK3 recognizes as epitope of CD4 blocked by binding of HIV/SIV envelope to CD4. Auto-antibodies to CD4 in the serum of anti-CD4 treated macaques mask the SK3 epitope, but not the OKT4 epitope of CD4. No CD4 blocking activity was detected in the serum of HIVIG treated macaques.

FIG. 4 is an diagram illustrating the results of a comparison of the abilities of HIV-1 envelope protein (HIV-1 IIIB gp120), TRX1 (therapeutic anti-CD4 mAb) and serum CD4-autoantibodies (206.1 serum CD4 auto-antibodies) to inhibit staining with the CD4 mAbs OKT4, V4, M-T477, SK3, 7E14 and L120.

FIG. 5 shows the inhibition of CD4 staining with anti-CD4 monoclonal antibody 7E14 after immunisation of unchallenged macaques with TRX1.

FIG. 6 is an alignment of CD4 amino acid sequences from human, cynomolgus macaque, Rhesus macaque, pig-tailed macaque, Japanese macaque and chimpanzee.

### BRIEF DESCRIPTION OF THE SEQUENCES

SEQ ID NO: 1 is the amino acid sequence of human CD4.

SEQ ID NO: 2 is the amino acid sequence of cynomolgus macaque CD4.

SEQ ID NO: 3 is the amino acid sequence of Rhesus macaque CD4.

SEQ ID NO: 4 is the amino acid sequence of pig-tailed macaque CD4.

SEQ ID NO: 5 is the amino acid sequence of Japanese macaque CD4.

SEQ ID NO: 6 is the amino acid sequence of chimpanzee CD4.

### DETAILED DESCRIPTION OF THE INVENTION

The present invention provides a method for generating anti-CD4 auto-antibodies comprising administering to an individual an anti-CD4 antibody molecule wherein administration of said antibody molecule results in the generation of anti-CD4 auto-antibodies that block binding of HIV to CD4.

The anti-CD4 antibody molecule typically has a low affinity for native CD4 in the individual. Administration of low affinity anti-CD4 antibodies results in the generation of anti-idiotypic antibodies which in turn lead to the generation of anti-anti-idiotypic antibodies, which are capable of binding to native CD4 in the individual, i.e. are anti-CD4 auto-antibodies.

Anti-CD4 auto-antibodies may also be generated by administering to an individual a CD4 protein, or peptide fragment thereof, which differs from native CD4 in the individual. In this embodiment, the CD4 auto-antibodies are generated to an epitope within the CD4 protein or peptide, which epitope is similar but not identical to an epitope in native CD4 in the individual. The CD4 protein may be one from a different species to the individual. For example, where the individual is human, the CD4 protein may be from a non-human primate. Alternatively, the CD4 protein may be a mutant CD4 protein as described herein.

The affinity of the anti-CD4 auto-antibodies generated in the individual may be greater than the affinity of the anti-CD4 antibody used to generate the auto-antibodies. The anti-CD4 auto-antibodies are generally of low avidity. Thus, the auto-antibody response may be distinguished from the anti-CD4 antibody used to vaccinate the individual not only in that binding to CD4 is reversible and anti-CD4 auto-antibodies can be washed off cells but also their affinity is sufficient that they can block binding of anti-CD4 monoclonal antibodies and, preferably, HIV envelope. CD4 cell counts are stable in vaccinated individuals that have these low affinity anti-CD4 auto-antibodies. Thus, the CD4 auto-antibodies do not appear to cause lysis of CD4+ cells through complement or antibody dependent cellular cytotoxicity (ADCC). Binding of the anti-CD4 auto-antibodies to CD4 on CD4+ lymphocytes blocks the binding site for HIV but does not cause lysis of the CD4+ lymphocytes.

### Antibodies

The anti-CD4 antibody molecules administered to the individual typically bind to CD4 of the individual with a low affinity. High affinity binding of an anti-CD4 antibody to CD4 in vivo may modify CD4+ T-cell responses and be immunosuppressive. If high affinity anti-CD4 antibody molecules were used, most would be bound to cellular CD4 and would be unavailable for immunization, i.e. the idiotype would not be presented to the immune system so no anti-idiotype antibodies and hence no anti-anti-idiotype CD4 auto-antibodies would be generated. Also, the anti-idiotype antibody response to a high affinity anti-CD4 antibody molecule may too closely resemble the native CD4 epitope (too close to self) preventing generation of the desired CD4 auto-antibody response (anti-anti-idiotype). It is unlikely that high affinity and high avidity auto-antibodies to CD4 that could fix complement would be generated, since during T cell development, auto-reactive clones are deleted to prevent such strong anti-self reactions. The method of the invention thus generates a high affinity/avidity antibody response to a CD4-like epitope which can cross-react with the native CD4 epitope, but with low avidity, preventing complement fixation and allowing self tolerance to be circumvented.

Accordingly, the low affinity anti-CD4 antibody molecule is one which is available for generating antibodies to its Fab, rather than being bound to CD4+ cells, and which modifies CD4+ T cell responses.

Low affinity binding of an anti-CD4 antibody molecule to CD4 may be detected by determining the ability of the anti-CD4 antibody molecule to block or reduce staining using anti-CD4 monoclonal antibodies that recognize the same region of CD4. Suitable methods are described in the Examples. Typically, binding of a low affinity anti-CD4 antibody molecule to CD4 from the individual can not be detected directly as the low affinity anti-CD4 antibody molecules would be washed off in subsequent secondary staining steps. CD4 antibody molecule binding to CD4, for example recombinant human CD4, may also be detected on a chip using a BIAcore instrument which is capable of measuring low avidity/affinity interactions.

A high affinity antibody will typically give around 1500 response units in BIAcore analysis after washing. A low affinity antibody typically gives a lower response. For example, a low affinity antibody may give from about 50 to about 250 response units, such as about 100, about 150 or about 200 response units.

Low affinity anti-CD4 antibodies and antibody molecules may be obtained by any suitable method. For example, suitable antibodies may be generated to a CD4-like epitope. A CD4-like epitope is an epitope which is not present in native CD4 in the individual to whom the anti-CD4 antibodies are administered, but which is similar to an epitope present in CD4 of the individual. That is, a CD4-like epitope is one which is similar to but not identical to an epitope in native CD4 in the individual.

The CD4-like epitope may be one which is present in a CD4 protein from a different species to the individual in which the auto-antibody response is required. Where the individual is a human, the anti-CD4 antibody may be specific for a CD4-like epitope in a non-human primate CD4 protein, such as CD4 from a macaque. Thus, the anti-CD4 antibody for administration to a human may be generated using a non-human primate CD4 protein, or peptide fragment thereof, for example macaque CD4. Conversely, where the individual is a non-human primate, the CD4-like epitope may be from human CD4, or from CD4 derived a different species of non-human primate.

Means for preparing and characterising antibodies are well known in the art, see for example Harlow and Lane (1988) “Antibodies: A Laboratory Manual”, Cold Spring Harbor Laboratory Press, Cold Spring Harbor, N.Y.

To generate suitable antibodies for administration to an individual, recombinant CD4 from a different species may be used to vaccinate mice in order to make a range of anti-CD4 monoclonal antibodies. These monoclonal antibodies may then be screened to select a monoclonal antibody to an epitope in the recombinant CD4 which has low affinity for the corresponding CD4 epitope in the individual. For example, where the individual is human the recombinant CD4 may be macaque CD4 or vice versa.

The CD4-like epitope may be present in a mutant CD4 protein. Thus, another example of antibodies that may be used to produce an auto-antibody response are antibodies directed to a CD4 epitope from the individual in which the auto-antibody response is required, which epitope is mutated or conformationally altered. The epitope may be conformationally altered by one or more amino acid mutation in a region of CD4 outside the epitope itself. The mutation may be a deletion, substitution or addition mutation that affects the conformation of the CD4 protein. The antibodies may be generated using a peptide fragment or peptide mimic of CD4 which comprises or consists of the epitope.

In a mutant CD4 molecule used to generate anti-CD4 antibodies for vaccination, the amino acid differences with the CD4 molecule in the individual are typically small. For example, 1, 2, 3, 4, 5, 8, 10 or 15 amino acids may be added, deleted or substituted in the mutant CD4 molecule. The CD4-like epitope may, for example, be a peptide epitope which comprises 1, 2, 3 or more amino acid substitutions, deletions and/or additions.

The anti-CD4 antibody typically blocks the HIV binding site on CD4. The epitope to which the anti-CD4 antibody binds is preferably located in the CD4 molecule such that binding of the low affinity anti-CD4 antibody to CD4 would obscure the binding site for HIV on CD4. For example, the epitope may be present in or close to the HIV binding site in CD4.

The binding site for the HIV envelope protein on CD4 has been localized to the V₁ domain of CD4. Residues 41-52 of CD4 have been implicated in binding of HIV envelope protein (Jameson et al., 1988; Sattentau et al., 1989). Domains V₁ and V₂ lie in close spatial proximity and antibodies that recognize epitopes that span both V₁ and V₂ can also inhibit HIV envelope binding to CD4. Changes in V₂ have also been reported to affect V₁, possibly through conformational changes, resulting in reduced binding affinity of HIV-1 envelope.

There are a number anti-V₁ CD4 antibodies, including Leu3a (also known as SK3), that cross block HIV-1 envelope binding (Sattentau et al., 1989). Leu3a maps to the HIV-1 gp120 binding site of CD4 and OKT4 maps to a site independent of HIV-1 gp120 binding (Kunkl et al., 1994). The antibody 7E14 has also maps to the HIV-1 gp120 binding site of CD4.

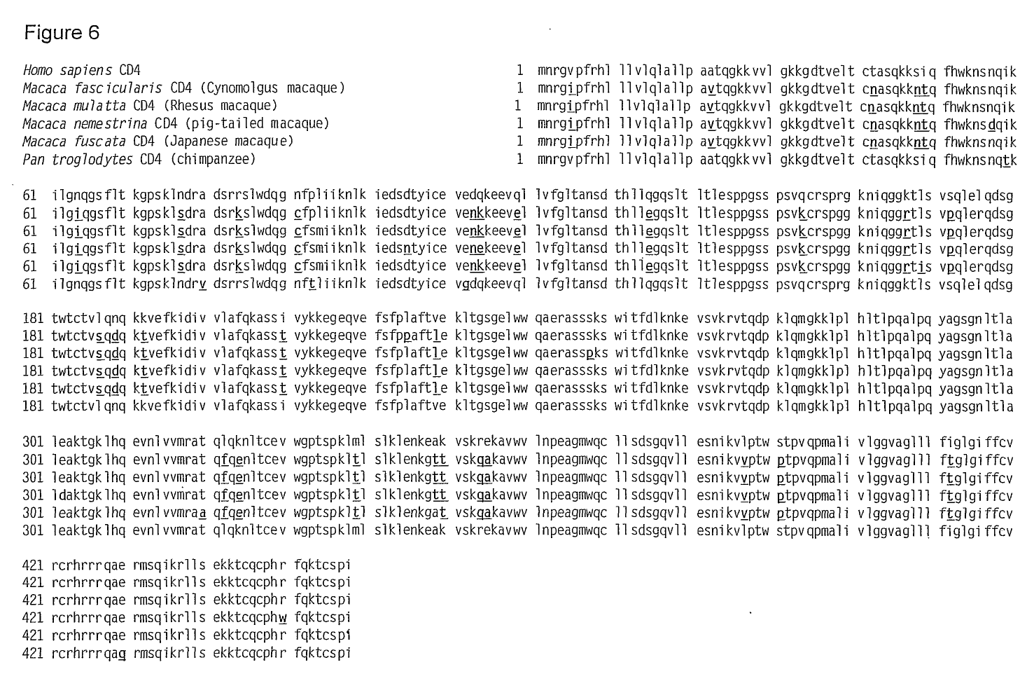

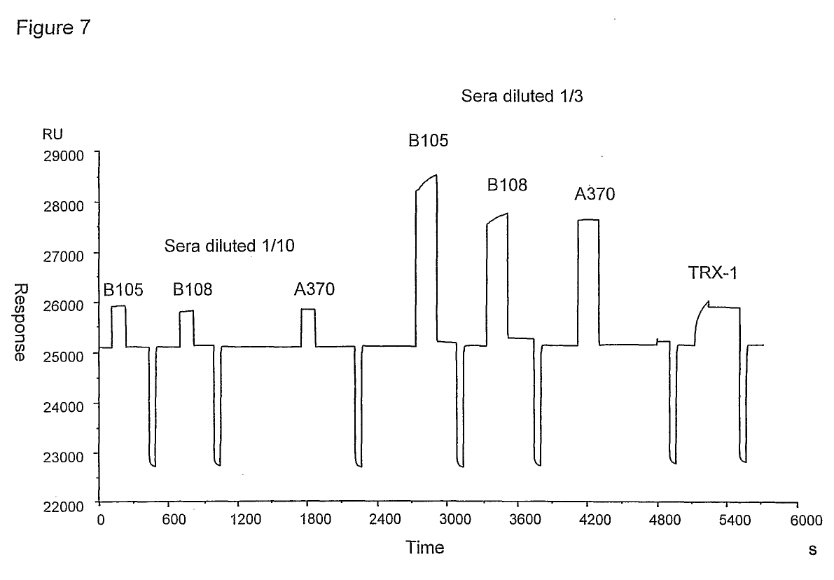

Comparison of human and macaque CD4 sequences are shown in FIG. 6. Amino acid residues 42 (Thr to Asn), 48 (Ser to Asn) and 49 (Ile to Thr) lie within the region of CD4 implicated in binding HIV envelope protein and are likely candidates for causing the observed low affinity of anti-human CD4 monoclonal antibody used in the Examples for macaque CD4. Accordingly, mutant CD4 proteins with mutations at one or more of positions 42, 48 and 49 are preferred.

Typically, the anti-CD4 antibody typically “specifically binds” or “is specific for” a CD4-like epitope, i.e. it binds with preferential affinity to a CD4-like epitope compared to binding to the equivalent epitope in CD4 in the individual. The anti-CD4 antibody typically has at least a 2 fold, 5 fold, 10 fold, 50 fold or 100 fold lower affinity for native CD4 in the individual in which the auto-antibody response is required compared to the affinity of the CD4 protein or peptide fragment used to generate the anti-CD4 antibody. The anti-CD4 antibody used for vaccination may be a whole antibody. The antibody typically has a mutation in the Fc region which prevents complement mediated lysis and antibody dependent cellular cytotoxicity (ADCC). Complement mediated cell lysis and ADCC may also be avoided by deletion of the Fc region from the antibody. Accordingly, antibody fragments may be used to generate the auto-antibody response. Suitable antibody fragments include Fab fragments, Fab₂ fragments, Fv fragments, single chain Fv (scFv) fragments and diabodies.

To test for complement mediated lysis, serum from a vaccinated individual may be mixed 1:1 with fresh whole blood from a naïve individual and incubated for 2 hours at 37° C. Lysis of CD4+ cells can then be assessed by flow cytometry to determine whether there has been a reduction in total CD4+ cell count or using a haematology analyser to determine whether there has been a reduction in total lymphocyte counts. To assess ADCC the same assay would be used but the blood would be incubated for from 24 to 72 hours. A reduction in CD4+ cell counts and/or total lymphocyte counts during vaccination would indicate CD4+ cell lysis.

Fab fragments have the advantage that they may have a lower affinity/avidity than Fab₂ fragments or whole antibody. Thus, an anti-CD4 auto-antibody response may be generated using a Fab fragment which is generated to CD4 from the individual in which the auto-antibody response is required.

The antibody molecule may be a recombinant antibody. The term recombinant antibody is intended to include all antibodies that are prepared, expressed, created or isolated by recombinant means, such as antibodies expressed using a recombinant expression vector transfected into a host cell; antibodies isolated from a recombinant, combinatorial antibody library; antibodies isolated from an animal (e.g. a mouse) that is transgenic for human immunoglobulin genes; or antibodies prepared, expressed, created or isolated by any other means that involves splicing of human immunoglobulin gene sequences to other DNA sequences. Such recombinant antibodies include humanised, CDR grafted, chimeric, deimmunised and in vitro generated antibodies. Preferably the antibody is humanized to reduce unwanted immunogenicity and focus the immune response on the Fab to make anti-idiotype responses.

Preferably the anti-CD4 antibody is a monoclonal antibody. However, polyclonal antibodies may also be used.

The antibody may be conjugated to a functional moiety such as a drug, detectable moiety or a solid support.

Two or more low affinity anti-CD4 antibody molecules may be administered in combination to generate an anti-CD4 auto-antibody response. The two or more anti-CD4 antibody molecules may be directed to the same or different epitopes. Bispecific or multivalent antibody molecules which bind to two or more different CD4-like epitopes may also be used to generate a CD4 auto-antibody response.

### Individual

The individual is a mammal, preferably a primate. The primate may be human or a non-human primate. The non-human primate is preferably one which is used in experimental studies and which is purpose-bred for laboratory use. The non-human primate for laboratory use is typically one that is used in studies relating to HIV/SIV infection. The non-human primate may be a macaque, preferably a cynomolgus macaque.

The human individual may be an individual at risk of HIV infection. In an alternative, the individual may be infected with HIV. Typically the individual is in the early stages of HIV infection but the human individual may be an individual who is suffering from AIDS.

### Prophylaxis

The auto-antibody response may be used to prevent or inhibit HIV infection in humans or SIV infection in non-human primates. Accordingly, the invention provided a method of vaccinating against HIV and/or SIV, which method comprises administering to an individual a low affinity anti-CD4 antibody as defined herein or a CD4 protein, or peptide fragment thereof, which is different to native CD4 in the individual. A vaccine comprising an anti-CD4 antibody, a pharmaceutically acceptable carrier or diluent and, optionally, an adjuvant is also provided.

Administration of the antibody, protein or peptide to the individual results in the generation of an anti-CD4 auto-antibody response. The auto-antibodies bind reversibly to native CD4 such that binding of HIV and/or SIV to CD4 is inhibited, thus preventing HIV and/or SIV entry into CD4+ cells.

In a method of vaccination according to the invention a prophylactically effective amount of an anti-CD4 antibody-molecule or CD4 protein or peptide with low affinity for native CD4 in the individual being vaccinated is administered to the individual.

A prophylactically effective amount is which is effective, upon single- or multiple-dose administration to an individual, in preventing or delaying HIV or SIV infection. Infection is delayed by inhibiting entry of the virus into cells of the individual.

The prevention or delay of HIV or SIV infection may be determined by any suitable method. For example, plasma vRNA loads may be used to quantitate levels of virus replication. DNA PCR and serology can also be used to determine protection against infection.

The effectiveness of the vaccine may also be determined by monitoring the level of CD4 auto-antibodies elicited. The level of CD4 auto-antibodies elicited through vaccination may, for example, be quantitated based on their ability to reduce the mean fluorescence intensity (MFI) of staining of anti-CD4 mAbs to the HIV binding site of CD4.

### Therapy

The antibody response is also useful in the treatment of HIV/SIV infection, i.e. vaccination to generate an auto-antibody response to CD4 may be a therapeutic vaccine. Thus, the method of generating auto-antibodies is useful in preventing or delaying the outset of AIDS in HIV infected individuals.

The use of a therapeutic vaccine in accordance with the present invention has the advantage over using humanized anti-CD4 therapeutic antibodies themselves as anti-retroviral agents in that vaccinates would produce their own anti-CD4 auto-antibodies and patients would not need to receive regular injections of therapeutic antibody. Accordingly, the invention provides a method of treating an HIV or SIV infected individual, which method comprises administering to the infected individual an amount of an anti-CD4 antibody or CD4 protein, or fragment thereof, as defined herein effective to elicit CD4 auto-antibodies in the individual. A pharmaceutical composition comprising an anti-CD4 antibody or CD4 protein, or fragment thereof, as defined herein and a pharmaceutically acceptable carrier or diluent is also provided.

In a therapeutic method of the invention, a therapeutically effective amount of an anti-CD4 antibody molecule with low affinity for native CD4, or a CD4 protein or peptide, is administered to an infected individual. A therapeutically effective amount is an amount of the anti-CD4 antibody, protein or peptide which is effective, upon single or multiple dose administration to a individual in curing, alleviating, relieving or improving the condition of an infected individual beyond that expected in the absence of such treatment. A therapeutically effective amount of antibody, protein or peptide in an amount effective to generate an auto-antibody response that inhibits entry of HIV into CD4+ cells in the individual.

The effectiveness of the therapeutic treatment may be determined by any suitable method. For example, the level of CD4-auto-antibodies elicited in the individual may be determined. Improvements in CD4+ cell counts may be used to indicate an overall improvement in health due to lower viral replication.

### Administration

Formulation with standard pharmaceutically acceptable carriers and/or diluents may be carried out using routine methods in the pharmaceutical art. For example, the antibody molecule or CD4 protein/peptide may be dissolved in physiological saline or water for injections. The exact nature of a formulation will depend upon several factors including the desired route of administration. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The antibody molecule or CD4 protein/peptide is typically administered by a parenteral route. The anti-CD4 antibody may be administered subcutaneously, intravenously, intramuscularly, intraperitoneally, intrasternally, transdermally or by infusion techniques. It is not essential to use an adjuvant when the antibody is for intravenous or intraperitoneal administration. Subcutaneous or intramuscular administration typically require use of an adjuvant. Subcutaneous or intramuscular administration may be used to boost following initial administration of the antibody via an intravenous or intraperitoneal route in order to reduce the risk of anaphylactic shock.

Vaccines and pharmaceutical compositions may be prepared from one or more of the antibody molecules, proteins or peptides defined herein and a physiologically acceptable carrier or diluent. Typically, such vaccines are prepared as injectables, either as liquid solutions or suspensions; solid forms suitable for solution in, or suspension in, liquid prior to injection may also be prepared. The preparation may also be emulsified, or the protein encapsulated in a liposome. The active immunogenic ingredient may be mixed with a carrier or diluent which is pharmaceutically acceptable and compatible with the active ingredient. Suitable carriers and diluents are, for example, sterile water, saline, PBS, dextrose, glycerol, ethanol, or the like and combinations thereof. Preferably the vaccine or pharmaceutical composition is in the form of a sterile, aqueous, isotonic saline solution. No carrier protein is required, but a carrier protein may be used if desired.

In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of auto-antibodies directed against CD4 resulting from administration of the anti-CD4 antibody molecule or CD4 protein/peptide in vaccines which are also comprised of the various adjuvants.

The vaccines are conventionally administered parentally, by injection, for example, either subcutaneously, intracutaneously or intramuscularly. The vaccines may alternatively be administered by local administration to the skin, such as by intracutaneous or subcutaneous administration. Additional formulations which are suitable for other modes of administration include suppositories, oral formulations and formulations for transdermal administration. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

Vaccine compositions suitable for delivery by needleless injection, for example, transdermally, may also be used.

The proteins or peptides as defined herein may be formulated into the vaccine as neutral or salt forms. Pharmaceutically acceptable salts include the acid addition salt (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

The dose may be determined according to various parameters, especially according to the age, weight and condition of the patient to be treated; the route of administration; and the required regimen. A physician will be able to determine the required route of administration and dosage for any particular patient.

The vaccines are administered in a manner compatible with the dosage formulation and in such amount will be prophylactically and/or therapeutically effective. The quantity to be administered, which is generally in the range of 5 μg to 100 mg, preferably 250 μg to 10 mg, more preferably from 1 mg to 3 mg, of antigen per dose, depends on the subject individual to be treated, capacity of the individual's immune system to synthesize antibodies, and the degree of protection desired. Precise amounts of active ingredient required to be administered may depend on the judgement of the practitioner and may be peculiar to each individual.

The vaccine may be given in a single dose schedule, or preferably in a multiple dose schedule. A multiple does schedule is one in which a primary course of vaccination may be 1 to 10 separate doses, for example 3, 4, 5, 6, 7 or 8, followed by other doses given at subsequent time intervals required to maintain and or reinforce the immune response, for example at 1 to 4 months for a second dose, and if needed, a subsequent dose(s) after several months. The dosage regimen will also, at least in part, be determined by the need of the individual and be dependent upon the judgement of the practitioner.

An exemplary, non-limiting range for a therapeutically or prophylactically effective amount of an antibody of the invention is 0.1-20 mg/kg, more preferably 1-10 mg/kg. The antibody can be administered by intravenous infusion at a rate of less than 30, 20, 10, 5 or 1 mg/min to reach a dose of about 1 to 100 mg/m² or about 5 to 30 mg/m². For antibody fragments which have lower molecular weights than a whole antibody, appropriate amounts can be proportionally less. Dosage values may vary with the severity of the condition of the individual.

For example, up to a total of about 10 mg per ml anti-CD4 antibody may be used for intravenous administration. For subcutaneous or intramuscular boosting with adjuvant, a total of, for example, 100 μg may be administered.

The present invention is described with reference to the following, non-limiting Examples:

### EXAMPLES

### Materials and Methods

### Animals

A total of 8 naïve, D-type retrovirus free, juvenile, purpose-bred cynomolgus macaques (Macaca fascicularis) were used in each of Examples 1 and 2. Macaques were housed and maintained in accordance with United Kingdom Home Office guidelines for the care and maintenance of non-human primates.

### Virus

In Example 1, all macaques, A363 to A370, were challenged intravenously on day 0 with 10 MID₅₀ of the pathogenic SIVmacJ5M (Stebbings et al., 2002).

### Detection of CD4 Auto-Antibodies

Specific auto-antibodies to CD4 were detected by inhibition of staining with the RPE conjugated anti-human CD4 mAbs 7E14 (Serotec Ltd, Oxford, UK) and SK3 (BD Biosciences, Oxford, UK), and failure to inhibit staining with the FITC conjugated anti-human CD4 mAbs OKT4 (Ortho Diagnostics) and V4 (BD Biosciences, Oxford, UK). Briefly, 200 μl of whole blood was incubated with conjugated anti-human CD4 mAbs in Sarstedt tubes for 5 minutes at room temperature on an orbital shaker. After staining, 1 ml of FACS lysis buffer (BD Biosciences, Oxford, UK) was added to each tube. Following lysis of red blood cells, lymphocytes were washed twice in PBS containing 2% FCS and 0.05% w/v sodium azide and then fixed in 2% paraformaldehyde in PBS. Lymphocyte CD4 staining was acquired using a FACSCalibur flow cytometer (BD Bioscience, Oxford, UK) and analysed using the Cell Quest program (BD Biosciences, Oxford, UK). CD4 positive cells were gated on the OKT4 stained population and the median fluorescent intensity (MFI) of staining with 7E14 or SK3 measured.

In Example 1, detection of specific auto-antibodies to CD4 in the heat-inactivated serum was accomplished by pre-incubating with naïve PBMC for 5 minutes, followed by addition of conjugated anti-CD4 mAbs and staining for 5 minutes as detailed above. PBMC from naïve macaques B1 to B4 were used to evaluate CD4 auto-antibody activity of serum from macaques A363 to A364.

Detection of auto-antibodies to CD4 using BIAcore was carried out using a human CD4 chip. Recombinant human CD4 was immobilised onto a BIAcore sensor chip by amine coupling. Binding of 10 μg/ml humanised anti-human CD4 mAB (TRX1) to the CD4 chip was determined and compared to binding of sera from control macaque A380 and macaques B105 and B108 from Example 2.

Serum levels of humanized anti-CD4 mAb are measure using an ELISA assay.

### Virus Detection and Quantification

The presence of SIV in PBMC or tissue samples was determined using SIV gag DNA PCR assays, as previously described (Rose et al., 1995). Levels of SIV RNA in plasma were determined at 14 days post wild type SIVmacJ5 challenge, as previously described (Clarke, Almond, and Berry, 2003). The sensitivity of the assay is 200 SIV RNA copies per ml of plasma.

### Example 1

### Therapeutic Anti-CD4 mAB Treatment

Macaques A363 to A366 were intravenously administered 10 mg of humanized anti-human CD4 mAb (TRX1) intravenously (Therapeutic Antibody Centre, Oxford, UK) on days −1, 0, 3, 6, 8, 10, 14 and 17. This humanized therapeutic anti-CD4 mAb carries a mutation in the Fc region which prevents complement mediated cell lysis and ADCC. This antibody has an approximately 10-100 fold lower affinity for macaque CD4 than human CD4. When this antibody was administered to humans previously, no CD4 auto-antibodies were observed.

Controls A367 to A370 were administered intravenously 10 mg of non-specific human intravenous immunoglobulin (HIVIG) on days −1, 0, 3, 6, 8, 10, 14 and 17. CD4 auto-antibodies were detected and SIV levels were determined as described above.

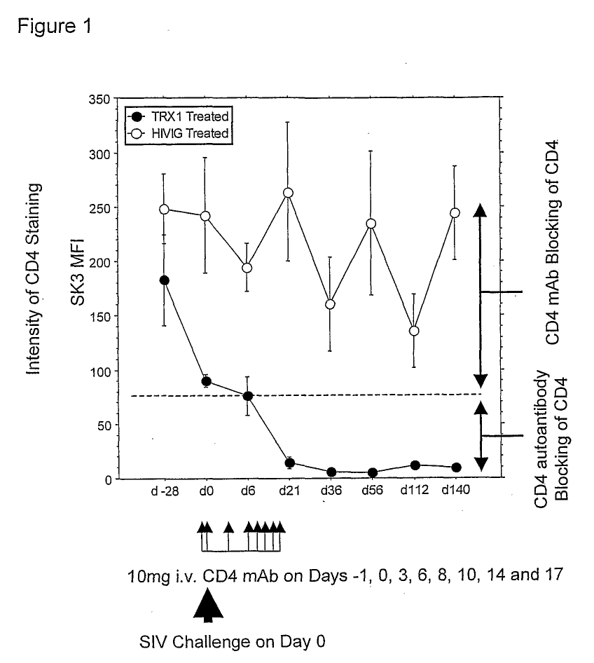

Following treatment with therapeutic anti-CD4 mAb 24 hrs earlier, significant (p=0.0277, paired t-test) inhibition of CD4 staining with the anti-CD4 mAb SK3 was observed by day 6 post SIVmacJ5 challenge (FIG. 1). An average 50% reduction in the MFI of SK3 staining was observed, which was maintained at day 6. No significant reduction in the MFI of SK3 staining was observed in HIVIG treated macaques.

By day 21, 4 days after the last treatment with therapeutic anti-human CD4 mAb, a further significant (p=0.0019, paired t-test) reduction in the MFI of SK3 staining compared to the day of SIVmacJ5 challenge was observed. This marked the appearance of CD4 auto-antibodies that inhibited CD4 mAb staining after anti-CD4 mAb therapy had stopped and were maintained up to day 140 when all macaques were terminated.

The increased affinity of this CD4 auto-antibody response distinguished it from anti-CD4 mAb therapy which was never able to achieve the degree of inhibition of SK3 staining observed.

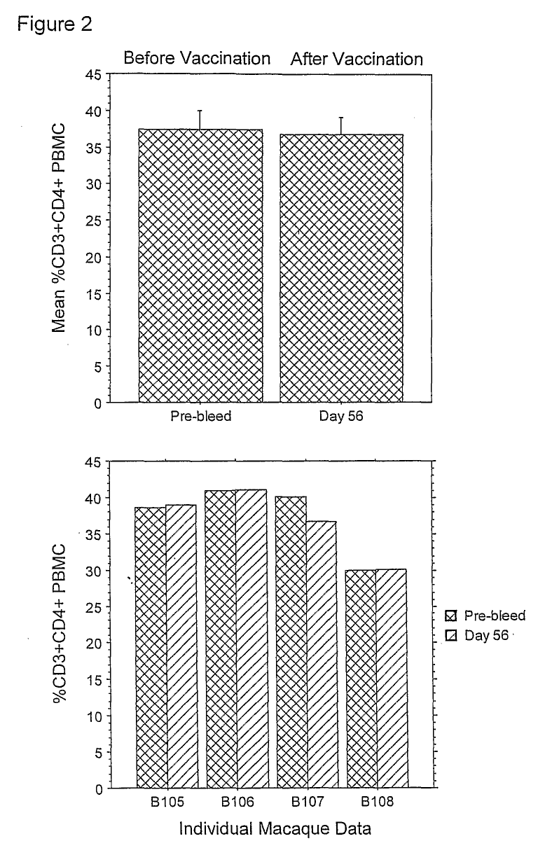

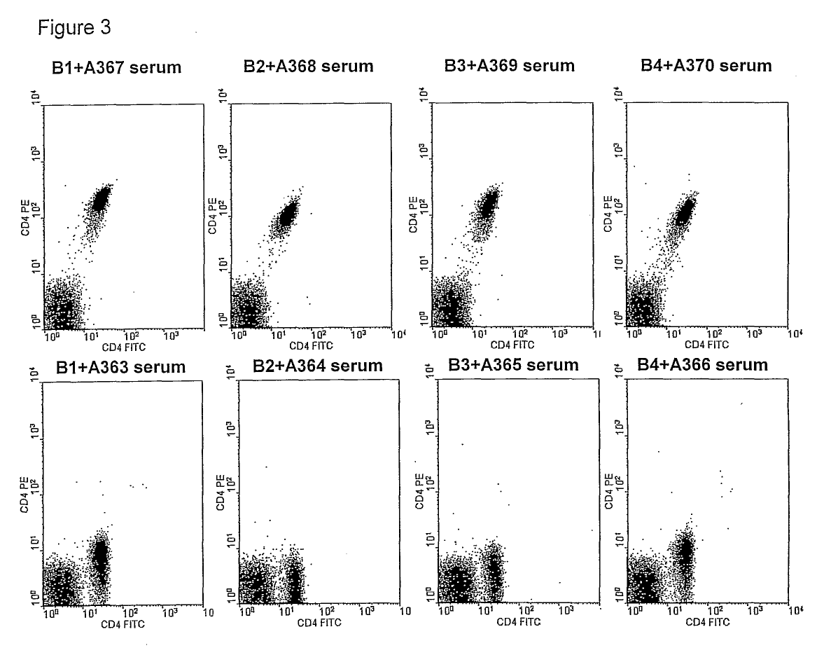

This CD4 auto-antibody does not cause lysis of CD4 lymphocytes (FIG. 2) and its ability to inhibit staining with anti-CD4 mAbs can be removed by washing prior to staining. Pre-incubation of naïve PBMC with serum from therapeutic anti-CD4 mAb treated animals inhibited subsequent CD4 mAb staining (FIG. 3). Serum from HIVIG treated animals did not inhibit subsequent CD4 mAb staining.

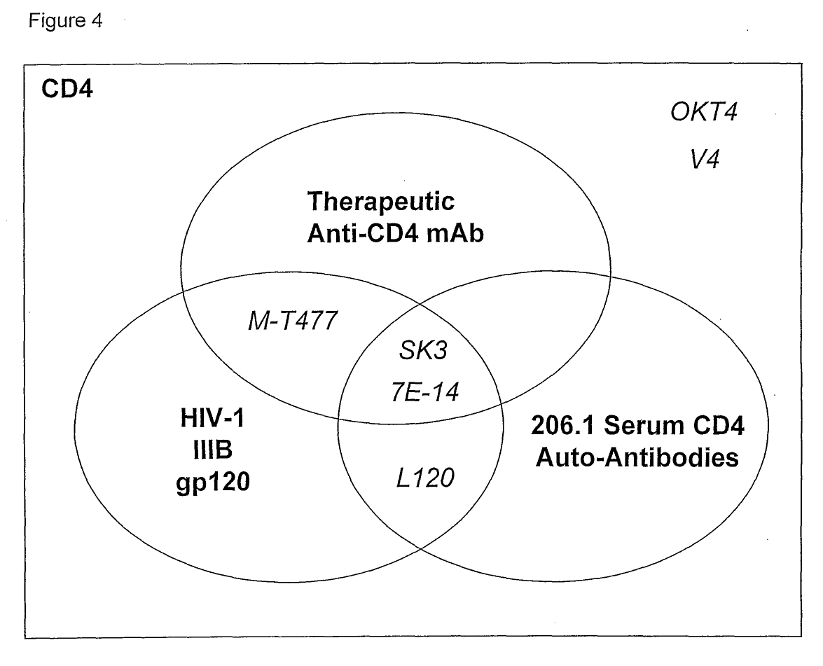

Comparison of the ability of HIV-1 envelope protein, therapeutic CD4 mAb and serum CD4 auto-antibodies to inhibit staining with the CD4 mAbs OKT4, V4, M-T477, SK3, 7E14 and L120 was assessed (FIG. 4). No inhibition of CD4 staining with the mAbs OKT4 or V4 was observed. HIV-1 envelope gp120 protein inhibits staining with the CD4 mAbs M-T477, SK3, 7E14 and L120. Serum CD4 auto-antibodies inhibited staining with the CD4 mAbs SK3, 7E14 and L120 whereas therapeutic CD4 mAb inhibited staining with the CD4 mAbs M-T477, SK3 and 7E14.

### Example 2

### Vaccination Using Anti-CD4 mAb

Macaques B105 to B108 were vaccinated intravenously with 10 mg of humanized anti-human CD4 mAb (TRX1) in 1 ml of PBS (Therapeutic Antibody Centre, Oxford, UK), on days −1, 0, 3, 6, 8, 10, 14 and 17. Macaques B109 to B112 were used as unvaccinated controls. CD4 auto-antibodies were detected as described above.

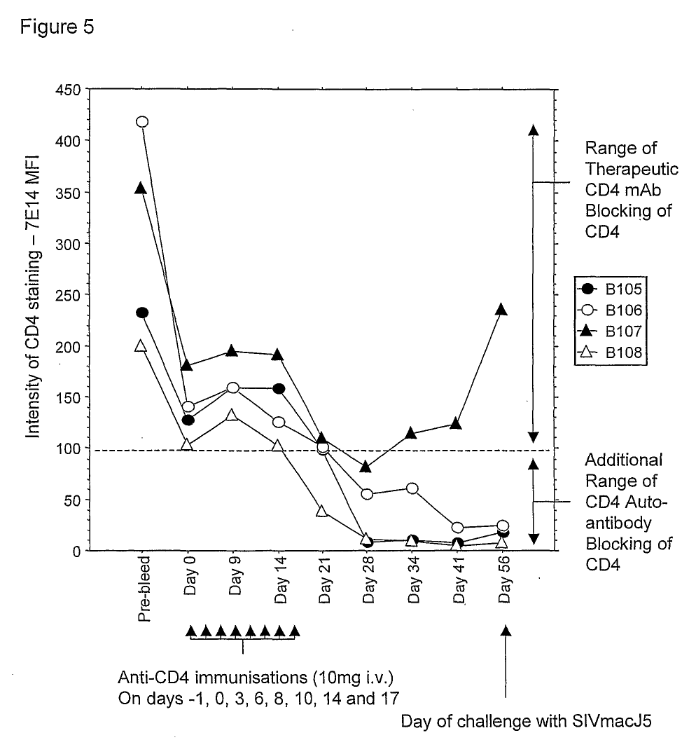

Inhibition of CD4 staining with anti-CD4 mAbs 7E14 was first observed on day 0, 24 hrs after the 1^{st} immunisation with therapeutic anti-human CD4 mAb (FIG. 5). Inhibition of CD4 staining with 7E14 was significant (p=0.0297, paired t-test), and resulted in an average 50% reduction in the MFI of 7E14 staining. This level of inhibition of 7E14 staining was maintained during the immunisation period with therapeutic anti-human CD4 mAb. By day 21, 4 days after the last immunisation with therapeutic anti-human CD4 mAb, a further significant (p=0.0174, paired t-test) reduction in 7E14 staining was observed, that marked the appearance of CD4 auto-antibodies. A further significant (p=0.0487, paired t-test) reduction in 7E14 MFI was observed at day 28, but no further reduction was observed at day 34. Inhibition of CD4 staining with the mAb SK3 was also confirmed at this time (data not shown). In particular, macaques B105 and B108 demonstrated complete inhibition of 7E14 staining. Macaques were challenged on day 56 with SIVmacJ5.

### Example 3

### BIAcore Analysis

The binding of sera from macaques B105 and B108 to human CD4 was analysed using a CD4 BIAcore chip. Sera from macaque A370 was used as a negative control and mAb TRX was used as a positive control. FIG. 7 is a sensorgram showing the BIAcore responses observed when the different sera/antibodies were added. On addition of sera, non-specific binding of serum proteins was observed prior to observation of specific low-affinity anti-CD4 auto-antibody binding for sera from B105 and B108 at a ⅓ dilution (curved line). The horizontal line for the control serum is consistent with the absence of any CD4 specific binding proteins. High affinity specific binding was observed following application of mAb TRX. The majority of TRX was retained on the chip after washing, whilst CD4 auto-antibodies in sera from B105 and B108 were rapidly eluted and only a small amount of auto-antibody binding remained after washing, consistent with low affinity interaction with CD4.

### REFERENCES

Jameson, B. A., Rao, P. E., Kong, L. I., Hahn, B. H., Shaw, G. M., Hood, L. E., and Kent, S. B. (1988). Location and chemical synthesis of a binding site for HIV-1 on the CD4 protein. Science 240(4857), 1335-9.
Kunkl, A., Valle, M. T., Fenoglio, D., Dodi, F., Morandi, N., Rizzo, F., and Manca, F. (1994). Detection of T cell CD4 epitopes in HIV-infected individuals. Eur J Histochem 38 Suppl 1, 41-6.
Sattentau, Q. J., Arthos, J., Deen, K., Hanna, N., Healey, D., Beverley, P. C., Sweet, R., and Truneh, A. (1989). Structural analysis of the human immunodeficiency virus-binding domain of CD4. Epitope mapping with site-directed mutants and anti-idiotypes. J Exp Med 170(4), 1319-34.
Clarke, S., Almond, N., and Berry, N. (2003). Simian immunodeficiency virus Nef gene regulates the production of 2-LTR circles in vivo. Virology 306(1), 100-8.
Rose, J., Silvera, P., Flanagan, B., Kitchin, P., and Almond, N. (1995). The development of PCR based assays for the detection and differentiation of simian immunodeficiency virus in vivo. J Virol Methods 51(2-3), 229-39.
Stebbings, R. J., Almond, N. M., Stott, E. J., Berry, N., Wade-Evans, A. M., Hull, R., Lines, J., Silvera, P., Sangster, R., Corcoran, T., Rose, J., and Walker, K. B. (2002). Mechanisms of protection induced by attenuated simian immunodeficiency virus. V. No evidence for lymphocyte-regulated cytokine responses upon rechallenge. Virology 296(2), 338-53.

1. A method of inhibiting entry of HIV into human cells, said method comprising administering to a human individual an anti-CD4 antibody molecule in an amount effective to elicit an anti-CD4 auto-antibody response in said individual, wherein binding of said anti-CD4 auto-antibodies to CD4 blocks the binding site for HIV.

2. A method according to claim 1, wherein said anti-CD4 antibody molecule is generated using a human CD4-like epitope.

3. A method according to claim 2, wherein said human CD4-like epitope is an epitope present in CD4 from a non-human primate.

4. A method according to claim 2, wherein said human CD4-like epitope is a mutant human CD4 epitope.

5. A method according to claim 1, wherein said anti-CD4 antibody molecule binds to human CD4 with a low affinity.

6. A method according to claim 5, wherein said anti-CD4 antibody binds to the same region of CD4 as HIV.

7. A method according to claim 6, wherein said anti-CD4 antibody molecule binds to the V1 region of CD4.

8. A method according to claim 1, wherein said anti-CD4 antibody molecule is one which competes for binding to human CD4 with one or more monoclonal antibody selected from the group consisting of SK3, 7E-14 and TRX.

9. A method according to claim 1, wherein said anti-CD4 antibody molecule is a whole antibody in which the Fc region is inactivated such that complement mediated cell lysis and/or antibody-dependent cell mediated toxicity is prevented.

10. A method according to claim 1, wherein said anti-CD4 antibody molecule is an antibody fragment which lacks an Fc region.

11. A method according to claim 10, wherein said antibody fragment is selected from a Fab or Fab₂ fragment.

12. A method according to claim 1, wherein said anti-CD4 auto-antibodies bind reversibly to CD4 such that binding of HIVgp120 to CD4 is inhibited.

13. A method of inhibiting entry of HIV into human cells, said method comprising administering to a human individual a protein or peptide comprising a human CD4-like epitope in an amount effective to elicit an anti-CD4 auto-antibody response in said individual, wherein binding of said anti-CD4 auto-antibodies to CD4 blocks the binding site for HIV.

14. A method according to claim 13, wherein said human CD4-like epitope is an epitope present in CD4 from a non-human primate.

15. A method according to claim 13, wherein said human CD4-like epitope is a mutant human CD4-epitope.

16. A method according to claim 13, wherein said human CD4-like epitope corresponds to an epitope in the region of human CD4 to which HIV binds.

17. A method according to claim 16, wherein said CD4-like epitope corresponds to an epitope in the V1 region of CD4.

18. A method for generating anti-CD4 auto-antibodies in a human individual comprising administering to an individual an anti-CD4 antibody molecule directed to a human CD4-like epitope, or a protein or peptide comprising a human CD4-like epitope, wherein administration of said antibody molecule, protein or peptide results in the generation of anti-CD4 auto-antibodies that block binding of HIV to CD4.

19. An anti-CD4 auto-antibody obtained by a method according to claim 18.

20. (canceled)

21. A pharmaceutical or vaccine composition for administration to a human individual, which composition comprises an anti-CD4 antibody molecule directed to a human CD4-like epitope, or a protein or peptide comprising a CD4-like epitope, together with a pharmaceutically acceptable carrier or diluent.

22. A vaccine composition according to claim 21 which further comprises an adjuvant.

23. (canceled)

24. A method of vaccinating against HIV comprising administering to a human individual an anti-CD4 antibody molecule or a protein or peptide comprising a CD4-like epitope in an amount effective to elicit an anti-CD4 auto-antibody response in said individual, wherein binding of said anti-CD4 auto-antibodies to CD4 blocks the binding site for HIV.

25. A method of preventing the onset of AIDS in a human individual infected with HIV, comprising administering to a human individual an anti-CD4 antibody molecule or a protein or peptide comprising a human CD4-like epitope in an amount effective to elicit an anti-CD4 auto-antibody response in said individual, wherein binding of said anti-CD4 auto-antibodies to CD4 blocks the binding site for HIV.

26. A method of inhibiting entry of SIV or SHIV into primate cells, said method comprising administering a primate individual an anti-CD4 antibody molecule in an amount effective to elicit an anti-CD4 auto-antibody response in said individual, wherein binding of said anti-CD4 auto-antibodies to CD4 blocks the binding site for SIV or SHIV.

27. A method of inhibiting entry of SIV or SHIV into primate cells, said method comprising administering to a primate individual a protein or peptide comprising a primate CD4-like epitope in an amount effective to elicit an anti-CD4 auto-antibody response in said individual, wherein binding of said anti-CD4 auto-antibodies to CD4 blocks the binding site for SIV or SHIV.


SEQUENCE LISTING (6 sequences)

SEQ ID NO: 1 - protein, 1264 aa, Human
METASNARGGLYVALPRPHEARGHISLELELEVALLEGLNLEALALELEPRALAALATHRGLNGLYLYSLYSVALVALLEGLYLYSLYSGLYASPTHRVALGLLETHRCYSTHRALASERGLNLYSLYSSERILEGLNPHEHISTRPLYSASNSERASNGLNILELYSILELEGLYASNGLNGLYSERPHELETHRLYSGLYPRSERLYSLEASNASPARGALAASPSERARGARGSERLETRPASPGLNGLYASNPHEPRLEILEILELYSASNLELYSILEGLASPSERASPTHRTYRILECYSGLVALGLASPGLNLYSGLGLVALGLNLELEVALPHEGLYLETHRALAASNSERASPTHRHISLELEGLNGLYGLNSERLETHRLETHRLEGLSERPRPRGLYSERSERPRSERVALGLNCYSARGSERPRARGGLYLYSASNILEGLNGLYGLYLYSTHRLESERVALSERGLNLEGLLEGLNASPSERGLYTHRTRPTHRCYSTHRVALLEGLNASNGLNLYSLYSVALGLPHELYSILEASPILEVALVALLEALAPHEGLNLYSALASERSERILEVALTYRLYSLYSGLGLYGLGLNVALGLPHESERPHEPRLEALAPHETHRVALGLLYSLETHRGLYSERGLYGLLETRPTRPGLNALAGLARGALASERSERSERLYSSERTRPILETHRPHEASPLELYSASNLYSGLVALSERVALLYSARGVALTHRGLNASPPRLYSLEGLNMETGLYLYSLYSLEPRLEHISLETHRLEPRGLNALALEPRGLNTYRALAGLYSERGLYASNLETHRLEALALEGLALALYSTHRGLYLYSLEHISGLNGLVALASNLEVALVALMETARGALATHRGLNLEGLNLYSASNLETHRCYSGLVALTRPGLYPRTHRSERPRLYSLEMETLESERLELYSLEGLASNLYSGLALALYSVALSERLYSARGGLLYSALAVALTRPVALLEASNPRGLALAGLYMETTRPGLNCYSLELESERASPSERGLYGLNVALLELEGLSERASNILELYSVALLEPRTHRTRPSERTHRPRVALGLNPRMETALALEILEVALLEGLYGLYVALALAGLYLELELEPHEILEGLYLEGLYILEPHEPHECYSVALARGCYSARGHISARGARGARGGLNALAGLARGMETSERGLNILELYSARGLELESERGLLYSLYSTHRCYSGLNCYSPRHISARGPHEGLNLYSTHRCYSSERPRILE

SEQ ID NO: 2 - protein, 1266 aa, Macaca fascicularis
METASNARGGLYILEPRPHEARGHISLELELEVALLEGLNLEALALELEPRALAVALTHRGLNGLYLYSLYSVALVALLEGLYLYSLYSGLYASPTHRVALGLLETHRCYSASNALASERGLNLYSLYSASNTHRGLNPHEHISTRPLYSASNSERASNGLNILELYSILELEGLYILEGLNGLYSERPHELETHRLYSGLYPRSERLYSLESERASPARGALAASPSERARGLYSSERLETRPASPGLNGLYCYSPHESERMETILEILELYSASNLELYSILEGLASPSERASPTHRTYRILECYSGLVALGLASNLYSLYSGLGLVALGLLELEVALPHEGLYLETHRALAASNSERASPTHRHISLELEGLGLYGLNSERLETHRLETHRLEGLSERPRPRGLYSERSERPRSERVALLYSCYSARGSERPRGLYGLYLYSASNILEGLNGLYGLYARGTHRLESERVALPRGLNLEGLARGGLNASPSERGLYTHRTRPTHRCYSTHRVALSERGLNASPGLNLYSTHRVALGLPHELYSILEASPILEVALVALLEALAPHEGLNLYSALASERSERTHRVALTYRLYSLYSGLGLYGLGLNVALGLPHESERPHEPRPRALAPHETHRLEGLLYSLETHRGLYSERGLYGLLETRPTRPGLNALAGLARGALASERSERSERLYSSERTRPILETHRPHEASPLELYSASNLYSGLVALSERVALLYSARGVALTHRGLNASPPRLYSLEGLNMETGLYLYSLYSLEPRLEHISLETHRLEPRGLNALALEPRGLNTYRALAGLYSERGLYASNLETHRLEALALEGLALALYSTHRGLYLYSLEHISGLNGLVALASNLEVALVALMETARGALATHRGLNPHEGLNGLASNLETHRCYSGLVALTRPGLYPRTHRSERPRLYSLETHRLESERLELYSLEGLASNLYSGLYTHRTHRVALSERLYSGLNALALYSALAVALTRPVALLEASNPRGLALAGLYMETTRPGLNCYSLELESERASPSERGLYGLNVALLELEGLSERASNILELYSVALVALPRTHRTRPPRTHRPRVALGLNPRMETALALEILEVALLEGLYGLYVALALAGLYLELELEPHETHRGLYLEGLYILEPHEPHECYSVALARGCYSARGHISARGARGARGGLNALAGLARGMETSERGLNILELYSARGLELESERGLLYSLYSTHRCYSGLNCYSPRHISARGPHEGLNLYSTHRCYSSERPRILE

SEQ ID NO: 3 - protein, 1265 aa, Macaca mulatta
METASNARGGLYILEPRPHEARGHISLELELEVALLEGLNLEALALELEPRALAVALTHRGLNGLYLYSLYSVALVALLEGLYLYSLYSGLYASPTHRVALGLLETHRCYSASNALASERGLNLYSLYSASNTHRGLNPHEHISTRPLYSASNSERASNGLNILELYSILELEGLYILEGLNGLYSERPHELETHRLYSGLYPRSERLYSLESERASPARGALAASPSERARGLYSSERLETRPASPGLNGLYCYSPHESERMETILEILELYSASNLELYSILEGLASPSERASPTHRTYRILECYSGLVALGLASNLYSLYSGLGLVALGLLELEVALPHEGLYLETHRALAASNSERASPTHRHISLELEGLGLYGLNSERLETHRLETHRLEGLSERPRPRGLYSERSERPRSERVALLYSCYSARGSERPRGLYGLYLYSASNILEGLNGLYGLYARGTHRLESERVALPRGLNLEGLARGGLNASPSERGLYTHRTRPTHRCYSTHRVALSERGLNASPGLNLYSTHRVALGLPHELYSILEASPILEVALVALLEALAPHEGLNLYSALASERSERTHRVALTYRLYSLYSGLGLYGLGLNVALGLPHESERPHEPRLEALAPHETHRLEGLLYSLETHRGLYSERGLYGLLETRPTRPGLNALAGLARGALASERSERPRLYSSERTRPILETHRPHEASPLELYSASNLYSGLVALSERVALLYSARGVALTHRGLNASPPRLYSLEGLNMETGLYLYSLYSLEPRLEHISLETHRLEPRGLNALALEPRGLNTYRALAGLYSERGLYASNLETHRLEALALEGLALALYSTHRGLYLYSLEHISGLNGLVALASNLEVALVALMETARGALATHRGLNPHEGLNGLASNLETHRCYSGLVALTRPGLYPRTHRSERPRLYSLETHRLESERLELYSLEGLASNLYSGLYTHRTHRVALSERLYSGLNALALYSALAVALTRPVALLEASNPRGLALAGLYMETTRPGLNCYSLELESERASPSERGLYGLNVALLELEGLSERASNILELYSVALVALPRTHRTRPPRTHRPRVALGLNPRMETALALEILEVALLEGLYGLYVALALAGLYLELELEPHETHRGLYLEGLYILEPHEPHECYSVALARGCYSARGHISARGARGARGGLNALAGLARGMETSERGLNILELYSARGLELESERGLLYSLYSTHRCYSGLNCYSPRHISARGPHEGLNLYSTHRCYSSERPRILE

SEQ ID NO: 4 - protein, 1266 aa, Macaca nemestrina
METASNARGGLYILEPRPHEARGHISLELELEVALLEGLNLEALALELEPRALAVALTHRGLNGLYLYSLYSVALVALLEGLYLYSLYSGLYASPTHRVALGLLETHRCYSASNALASERGLNLYSLYSASNTHRGLNPHEHISTRPLYSASNSERASPGLNILELYSILELEGLYILEGLNGLYSERPHELETHRLYSGLYPRSERLYSLESERASPARGALAASPSERARGLYSSERLETRPASPGLNGLYCYSPHESERMETILEILELYSASNLELYSILEGLASPSERASNTHRTYRILECYSGLVALGLASNGLLYSGLGLVALGLLELEVALPHEGLYLETHRALAASNSERASPTHRHISLELEGLGLYGLNSERLETHRLETHRLEGLSERPRPRGLYSERSERPRSERVALLYSCYSARGSERPRGLYGLYLYSASNILEGLNGLYGLYARGTHRLESERVALPRGLNLEGLARGGLNASPSERGLYTHRTRPTHRCYSTHRVALSERGLNASPGLNLYSTHRVALGLPHELYSILEASPILEVALVALLEALAPHEGLNLYSALASERSERTHRVALTYRLYSLYSGLGLYGLGLNVALGLPHESERPHEPRLEALAPHETHRLEGLLYSLETHRGLYSERGLYGLLETRPTRPGLNALAGLARGALASERSERSERLYSSERTRPILETHRPHEASPLELYSASNLYSGLVALSERVALLYSARGVALTHRGLNASPPRLYSLEGLNMETGLYLYSLYSLEPRLEHISLETHRLEPRGLNALALEPRGLNTYRALAGLYSERGLYASNLETHRLEALALEASPALALYSTHRGLYLYSLEHISGLNGLVALASNLEVALVALMETARGALATHRGLNPHEGLNGLASNLETHRCYSGLVALTRPGLYPRTHRSERPRLYSLETHRLESERLELYSLEGLASNLYSGLYTHRTHRVALSERLYSGLNALALYSALAVALTRPVALLEASNPRGLALAGLYMETTRPGLNCYSLELESERASPSERGLYGLNVALLELEGLSERASNILELYSVALVALPRTHRTRPPRTHRPRVALGLNPRMETALALEILEVALLEGLYGLYVALALAGLYLELELEPHETHRGLYLEGLYILEPHEPHECYSVALARGCYSARGHISARGARGARGGLNALAGLARGMETSERGLNILELYSARGLELESERGLLYSLYSTHRCYSGLNCYSPRHISTRPPHEGLNLYSTHRCYSSERPRILE

SEQ ID NO: 5 - protein, 1267 aa, Macaca fuscata
METASNARGGLYILEPRPHEARGHISLELELEVALLEGLNLEALALELEPRALAVALTHRGLNGLYLYSLYSVALVALLEGLYLYSLYSGLYASPTHRVALGLLETHRCYSASNALASERGLNLYSLYSASNTHRGLNPHEHISTRPLYSASNSERASNGLNILELYSILELEGLYILEGLNGLYSERPHELETHRLYSGLYPRSERLYSLESERASPARGALAASPSERARGLYSSERLETRPASPGLNGLYCYSPHESERMETILEILELYSASNLELYSILEGLASPSERASPTHRTYRILECYSGLVALGLASNLYSLYSGLGLVALGLLELEVALPHEGLYLETHRALAASNSERASPTHRHISLELEGLGLYGLNSERLETHRLETHRLEGLSERPRPRGLYSERSERPRSERVALLYSCYSARGSERPRGLYGLYLYSASNILEGLNGLYGLYARGTHRILESERVALPRGLNLEGLARGGLNASPSERGLYTHRTRPTHRCYSTHRVALSERGLNASPGLNLYSTHRVALGLPHELYSILEASPILEVALVALLEALAPHEGLNLYSALASERSERTHRVALTYRLYSLYSGLGLYGLGLNVALGLPHESERPHEPRLEALAPHETHRLEGLLYSLETHRGLYSERGLYGLLETRPTRPGLNALAGLARGALASERSERSERLYSSERTRPILETHRPHEASPLELYSASNLYSGLVALSERVALLYSARGVALTHRGLNASPPRLYSLEGLNMETGLYLYSLYSLEPRLEHISLETHRLEPRGLNALALEPRGLNTYRALAGLYSERGLYASNLETHRLEALALEGLALALYSTHRGLYLYSLEHISGLNGLVALASNLEVALVALMETARGALAALAGLNPHEGLNGLASNLETHRCYSGLVALTRPGLYPRTHRSERPRLYSLETHRLESERLELYSLEGLASNLYSGLYALATHRVALSERLYSGLNALALYSALAVALTRPVALLEASNPRGLALAGLYMETTRPGLNCYSLELESERASPSERGLYGLNVALLELEGLSERASNILELYSVALVALPRTHRTRPPRTHRPRVALGLNPRMETALALEILEVALLEGLYGLYVALALAGLYLELELEPHETHRGLYLEGLYILEPHEPHECYSVALARGCYSARGHISARGARGARGGLNALAGLARGMETSERGLNILELYSARGLELESERGLLYSLYSTHRCYSGLNCYSPRHISARGPHEGLNLYSTHRCYSSERPRILE

SEQ ID NO: 6 - protein, 1267 aa, Pan troglodytes
METASNARGGLYVALPRPHEARGHISLELELEVALLEGLNLEALALELEPRALAALATHRGLNGLYLYSLYSVALVALLEGLYLYSLYSGLYASPTHRVALGLLETHRCYSTHRALASERGLNLYSLYSSERILEGLNPHEHISTRPLYSASNSERASNGLNTHRLYSILELEGLYASNGLNGLYSERPHELETHRLYSGLYPRSERLYSLEASNASPARGVALASPSERARGARGSERLETRPASPGLNGLYASNPHETHRLEILEILELYSASNLELYSILEGLASPSERASPTHRTYRILECYSGLVALGLYASPGLNLYSGLGLVALGLNLELEVALPHEGLYLETHRALAASNSERASPTHRHISLELEGLNGLYGLNSERLETHRLETHRLEGLSERPRPRGLYSERSERPRSERVALGLNCYSARGSERPRARGGLYLYSASNILEGLNGLYGLYLYSTHRLESERVALSERGLNLEGLLEGLNASPSERGLYTHRTRPTHRCYSTHRVALLEGLNASNGLNLYSLYSVALGLPHELYSILEASPILEVALVALLEALAPHEGLNLYSALASERSERILEVALTYRLYSLYSGLGLYGLGLNVALGLPHESERPHEPRLEALAPHETHRVALGLLYSLETHRGLYSERGLYGLLETRPTRPGLNALAGLARGALASERSERSERLYSSERTRPILETHRPHEASPLELYSASNLYSGLVALSERVALLYSARGVALTHRGLNASPPRLYSLEGLNMETGLYLYSLYSLEPRLEHISLETHRLEPRGLNALALEPRGLNTYRALAGLYSERGLYASNLETHRLEALALEGLALALYSTHRGLYLYSLEHISGLNGLVALASNLEVALVALMETARGALATHRGLNLEGLNLYSASNLETHRCYSGLVALTRPGLYPRTHRSERPRLYSLEMETLESERLELYSLEGLASNLYSGLALALYSVALSERLYSARGGLLYSALAVALTRPVALLEASNPRGLALAGLYMETTRPGLNCYSLELESERASPSERGLYGLNVALLELEGLSERASNILELYSVALLEPRTHRTRPSERTHRPRVALGLNPRMETALALEILEVALLEGLYGLYVALALAGLYLELELEPHEILEGLYLEGLYILEPHEPHECYSVALARGCYSARGHISARGARGARGGLNALAGLNARGMETSERGLNILELYSARGLELESERGLLYSLYSTHRCYSGLNCYSPRHISARGPHEGLNLYSTHRCYSSERPRILE